(19) United States
(12) Patent Application Publication
(10) Pub. No.: US 2011/0247617 A1
(43) Pub. Date: Oct. 13, 2011

(54) MOUTHPIECE FOR AN INHALER

(76) Inventors: Thomas Nilsson, Bromma (SE); Martin Birath, Bromma (SE)

(21) Appl. No.: 13/140,663

(22) PCT Filed: Dec. 18, 2009
(86) PCT No.: PCT/EP2009/067484

§ 371 Date: Jun. 17, 2011

(30) Foreign Application Priority Data
Dec. 19, 2008 (EP) 08172405.6

(51) Int. Cl. A61M 15/00

(52) U.S. Cl. 128/203.12

(57) ABSTRACT

The invention relates to a mouthpiece for a medical device. The mouthpiece extends along a first axis from a rear portion to a front portion with a middle portion arranged therebetween. The rear portion is for attachment to a medical device and the front portion is for inserting into the mouth of a user. The middle portion has a cross section parallel with a second axis substantially perpendicular to the first axis. The cross section extends in a direction substantially parallel with a second axis from a first limit to a second limit and has a width substantially perpendicular to both the first and second axes. The cross section includes a divergent portion between the first limit and a widest limit, at which the width is a maximum, and a convergent portion between the widest limit and the second limit. The widest limit is a first distance from the first limit and a second distance from the second limit and the first distance is greater than the second.

The present invention relates to a mouthpiece for use with a medical device, in particular to a mouthpiece for use with an inhaler device.

A mouthpiece is an essential part of many medical devices which are designed to introduce a medicament or other substance into the body via the mouth. Typically mouthpieces for medical devices have simple geometric cross sections, most typically elliptical. A user provided with such a device, such as an inhaler, is typically instructed by a doctor or other medical practitioner in the proper use of the device.

Mouthpieces for inhalers in the art include, for example that shown in U.S. Pat. No. 7,131,441 and mouthpieces have also been used in conjunction with specially designed nosepieces in a delivery device so that a user can actuate the device by exhaling through their mouth into the device and have medication delivered via the nose, for example as shown in GB 2 448 193.

The present invention provides a mouthpiece for a medical device, the mouthpiece extending along a first axis from a rear portion to a front portion with a middle portion arranged therebetween, the rear portion for attachment to a medical device and the front portion and middle portion for inserting into the mouth of a user, the middle portion having a cross section substantially perpendicular to the first axis, the cross section extending in a direction substantially parallel with a second axis from a first limit to a second limit and having a width substantially perpendicular to both the first and second axes, the cross section including a divergent portion between the first limit and a widest limit, at which the width is a maximum, and a convergent portion between the widest limit and the second limit, the widest limit being a first distance from the first limit and a second distance from the second limit, the first distance being greater than the second distance.

This provides a mouthpiece which more closely matches the shape of the mouth of a user when the lips are pursed so as to form a seal around a mouthpiece, this shape also corresponds closely with the shape of the lips when forming the letter “O”. It has been found that this shape is a relaxed shape for the mouth.

Literature has suggested that major variations in the configuration of the upper airway during inhalation can be caused by the position of the tongue (reference: Touraj et. Al. Dynamic change of the Upper Airway during Inhaltion via Aerosol Delivery Devices, Journal of Aerosol Medicine, Vol 17, Number 4, 2004). It is suggested that by having fewer such variations in configuration a greater degree of repeatability of inhalation technique can be achieved which may improve desirable lung deposition and reduce deposition in the mouth and therefore can help to reduce side effects. Users of medical devices do not always follow the instructions provided for the use of a device and this can lead to variations in device performance.

The present invention seeks to address the problem of such variations by providing a mouthpiece whose outer shape encourages a user, when using the mouthpiece, to adopt a relaxed mouth shape and relaxed jaw which may assist in relaxing the tongue and therefore result in fewer variations in the shape of the upper airway which could result in the benefit suggested above.

A mouthpiece for a medical device is provided, the mouthpiece extending along a first axis from a rear portion to a front portion with a middle portion arranged therebetween, the rear portion for attachment to a medical device and the front portion for inserting into the mouth of a user, the front portion including an end wall which is set at an angle to the first axis such that a lower portion protrudes further along the first axis than an upper portion.

It has been found that this can create a more convenient or comfortable mouthpiece for a user and may result in the user relaxing the jaw which may reduce variations in configurations of the upper airway. The teeth of a user are often offset with the upper teeth protruding further than the lower teeth. The amount of such offset may vary between different patient populations. The lower portion of the mouthpiece protruding further than the upper portion means that a user could bite onto the mouthpiece, or at least rest their teeth on the mouthpiece without the need to insert additional material into the mouth.

The angle made between a line from the furthest protrusion of the upper portion to the furthest protrusion of the lower portion and the plane of the second axis and width may be between 8° and 15°.

A different mouthpiece for a medical device is also provided, the mouthpiece extending along a first axis from a rear portion to a front portion with a middle portion arranged therebetween, the rear portion for attachment to a medical device and the front portion for inserting into the mouth of a user, the rear portion including an upper lip stop and a lower lip stop which are offset along the first axis such that the lower lip stop is set further away from the front portion than the upper lip stop.

The offset between the upper and lower lip stops may be between 2 and 5 mm.

Investigations revealed that the upper and lower lips of a user are typically different sizes and providing offset lip stops can create a more comfortable mouthpiece. A lip stop is typically a change in gradient in the outer surface of the mouthpiece so that the lip rests against the stop. The change in gradient may result in the mouthpiece becoming more rapidly deeper from top to bottom in a direction away from the front portion. It is also suggested that the inclusion of such lip stops may result in an improved seal between the mouth of a user and the mouthpiece. An improved seal means that a user can relax their mouth more than would otherwise be possible while still maintaining an acceptable seal.

It should be understood that any two of these features, the offset lip stops, the angled front wall and the unequal cross section can be combined, or all three features combined in a single mouthpiece as each feature alone provides an improved mouthpiece, a combination of any two, or all three features is a particularly useful embodiment.

The rear portion of the mouthpiece is for attachment to a medical device. Part or all of the mouthpiece may be formed integrally with some or all of the medical device. The mouthpiece may be formed separately from the medical device and attached thereto using any suitable means, for example an adhesive, welded connection, a snap fit arrangement, a releasable coupling or any other suitable mechanical or other arrangement.

The medical device may be any device which includes a mouthpiece which a user places in their mouth, or which is placed in the mouth of a user by a third party. The medical device may be an inhaler from which a medicament can be inhaled. The medicament may be an aerosolised powder, an aerosolised liquid or other inhalable medicament.

The mouthpiece may include at least one medicament conduit extending from the rear portion to the front portion through which a medicament can pass. There may be other conduits extending partially or fully through the mouthpiece in addition to, or instead of a medicament conduit. Conduits may be used to carry medicament from the device towards the mouth of a user, for example the medicament conduit mentioned above, could be used to transmit gas from the mouth of the user into the device, or could be used to direct air or other gas so as to modify the gas flow within, or leaving, another conduit, for example to increase turbulence. The at least one medicament conduit may extend substantially centrally through the mouthpiece substantially parallel with the first axis. The conduit may have a circular or elliptical cross section, or its cross section may substantially match the cross section of the middle portion of the mouthpiece.

The mouthpiece may be substantially symmetrical about the second axis. This helps to produce a more natural shape for the mouthpiece and can help a user to create a good seal all the way around the periphery of the mouthpiece, which is particularly useful for a mouthpiece for an inhaler.

Within the divergent portion the width may consistently increase along the second axis which leads to a smoother outer periphery profile. It should be understood that a mouthpiece may include a region within the divergent portion where the width decreases, before increasing again along the second axis.

Within the convergent portion the width may consistently decrease along the second axis which leads to a smoother outer periphery profile. It should be understood that a mouthpiece may include a region within the convergent portion where the width increases, before decreasing again along the second axis.

The width of the cross section of the middle portion of the mouthpiece may be zero at the first and second limits.

The outer periphery of the cross section of the middle portion may be curved in at least one of the divergent portion and the convergent portion. The outer periphery may be advantageously substantially entirely curved with no discontinuities.

In one embodiment the outer periphery is substantially parallel with the width at the first and second limits and is substantially parallel with the second axis at the widest point.

In the convergent potion the width may reduce by at least half of the maximum width in the tenth of the distance between the maximum width limit and the second limit closest to the second limit. This changes the shape of the cross section from what could be described as a “kite” shape to a diamond shape.

It should be understood that throughout this specification and in the claims that follow, unless the context requires otherwise, the word “comprise”, or variations such as “comprises” or “comprising”, implies the inclusion of the stated integer or step, or group of integers or steps.

The invention will now be further described, by way of example only, with reference to the following drawings in which:

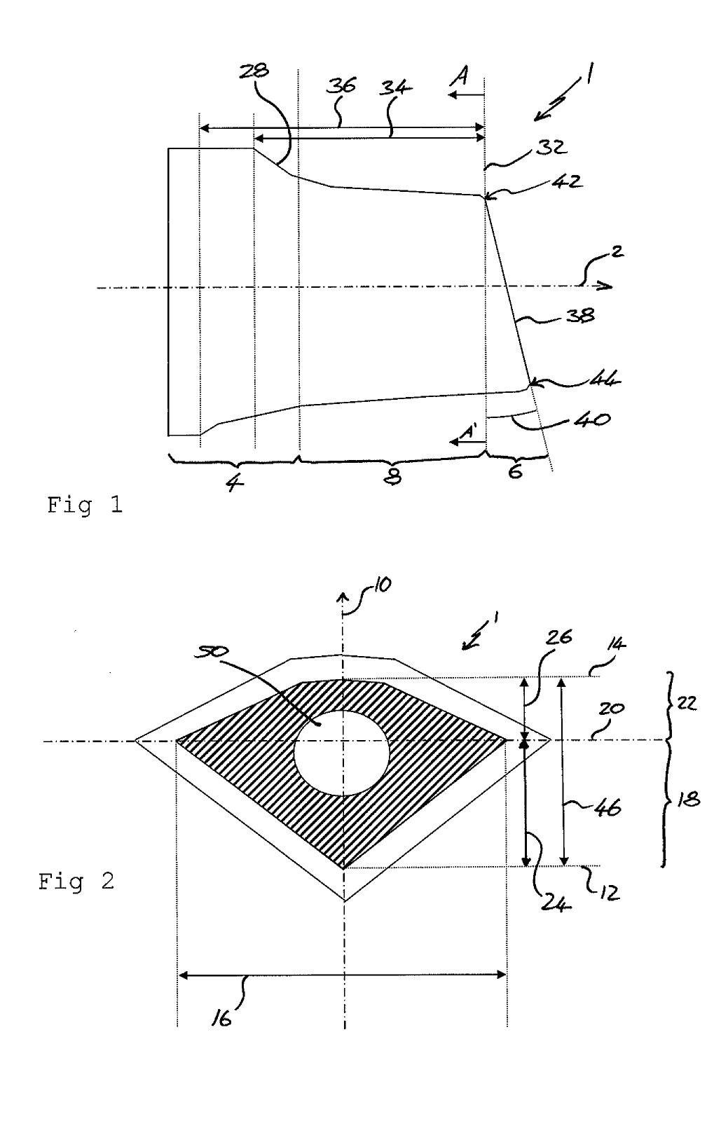

FIG. 1 shows a side view of a mouthpiece;

FIG. 2 shows a cross section through the middle portion of the mouthpiece of FIG. 1; and

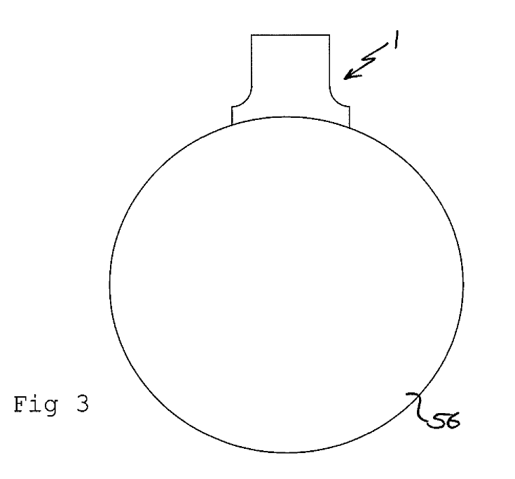

FIG. 3 shows a mouthpiece attached to a medical device.

FIGS. 1 and 2 show a mouthpiece 1 for a medical device (not shown in this FIG.). FIG. 1 shows a side view of the mouthpiece 1 and FIG. 2 shows a cross section view along the line A-A′ which is substantially perpendicular to a first axis 2. The mouthpiece 1 extends along a first axis 2 from a rear portion 4 to a front portion 6 with a middle portion 8 arranged therebetween. The rear portion 4 is suitable for attachment to a medical device and the front portion 6 is for inserting into the mouth of a user. The middle portion 8 has a cross section parallel with a second axis 10 substantially perpendicular to the first axis 2. The cross section extends in a direction substantially parallel with a second axis 10 from a first limit 12 to a second limit 14, the limits varying along the first axis 2.

The cross section has a width 16 substantially perpendicular to both the first and second axes 2,10. The cross section includes a divergent portion 18 between the first limit 12 and a widest limit 20, at which the width is a maximum, and a convergent portion 22 between the widest limit and the second limit 14. The widest limit 20 being a first distance 24 from the first limit and a second distance 26 from the second limit 14, first distance 24 being greater than the second 26.

The rear portion 4 includes an upper lip stop 28 and a lower lip stop 30. The lip stops 28,30 are offset along the first axis 2 such that the lower lip stop 30 is set further away from the front portion 6 than the upper lip stop 28. The lip stops 28,30 are defined by a change in gradient of the mouthpiece along the first axis 2. The change of gradient provides a surface change that a user can readily sense with their lips and against which the user can rest their lips. In this case the change in gradient provides an outwardly directed slope against which a user could rest their lips. It should be understood that the lip stops could be any suitable feature that a user could feel with their lips, for example a gradient change providing an outward slope and in FIG. 1, an inward slope, or dip, or simply a textured surface on which a user could rest their lips.

The upper lip stop 28 is set away from a rear limit 32 of the front portion 6 by an upper lip stop distance 34. The lower lip stop 30 is set away from a rear limit 32 of the front portion 6 by an lower lip stop distance 36.

The front portion 6 includes a front surface 38 which is set at an angle 40 relative to the first axis 2. It should be noted that the front surface may not be substantially planar, but that an upper portion 42 and a lower portion 44 are offset this angle 40.

A conduit 50 through the mouthpiece is shown as having a substantially circular cross section, but could be any suitable shape and arranged within the cross section substantially centrally as shown or offset in one or both directions within the cross section.

The mouthpiece may be provided in a variety of sizes, but three sizes are currently being considered, small (S), medium (M) and large (L). Suitable dimensions+/−10% are set forth in Table 1 with suitable ranges+/−10% shown in Table 2.

TABLE 1

| Dimension\Size | S | M | L |
|---|---|---|---|
| Max width 16 (mm) | 19.4 | 23.4 | 25.9 |
| Max height 46 (mm) | 11.5 | 13 | 17 |
| 1st Dist 24 (mm) | 7.6 | 8.6 | 11.1 |
| 2nd Dist 26 (mm) | 3.9 | 4.4 | 5.9 |
| Angle 40 (°) | 14.5 | 13 | 10 |
| Upper lip stop 34 (mm) | 14.1 | 14.1 | 14.1 |
| Lower lip stop 36 (mm) | 17.4 | 17.4 | 17.4 |

TABLE 2

| Dimension\Size | Minimum | Maximum |
|---|---|---|
| Max width 16 (mm) | 15 | 30 |
| Max height 46 (mm) | 10 | 20 |
| 1st Dist 24 (mm) | 6 | 14 |
| 2nd Dist 26 (mm) | 3 | 5 |
| Angle 40 (°) | 8 | 15 |
| Upper lip stop 34 (mm) | 10 | 17 |
| Lower lip stop 36 (mm) | 15 | 20 |

FIG. 3 shows the mouthpiece 1 attached to a medical device 56, in this case an inhaler for delivering an aerosolized medicament, for example a powder or liquid, to a user inhaling though the mouthpiece 1.

While in this example the features of offset lip stops, the angled front wall and the unequal cross section are combined, it should be understood that, since each feature alone provides an improved mouthpiece, the features can be used alone, in a combination of any two, or all three features combined together

It should be understood that the invention has been described above by way of example only and that modifications in detail can be made without departing from the scope of the claims.

1. A mouthpiece for a medical device, the mouthpiece extending along a first axis from a rear portion to a front portion with a middle portion arranged therebetween, the rear portion for attachment to a medical device and the front portion and middle portion for inserting into the mouth of a user, the middle portion having a cross section substantially perpendicular to the first axis, the cross section extending in a direction substantially parallel with a second axis from a first limit to a second limit and having a width substantially perpendicular to both the first and second axes, the cross section including a divergent portion between the first limit and a widest limit, at which the width is a maximum, and a convergent portion between the widest limit and the second limit, the widest limit being a first distance from the first limit and a second distance from the second limit, the first distance being greater than the second distance.

2. A mouthpiece as claimed in claim 1, in which the cross section of the middle portion is substantially symmetrical about the second axis.

3. A mouthpiece as claimed in claim 1, in which in the divergent portion the width increases along the second axis, and in the convergent portion the width decreases along the second axis.

4. A mouthpiece as claimed in claim 1, in which the width of the cross section of the middle portion is zero at the first and second limits.

5. A mouthpiece as claimed in claim 1, in which the cross section of the middle portion has an outer periphery and in at least one of the divergent portion and the convergent portion the outer periphery is curved.

6. A mouthpiece as claimed in claim 5, in which the width reduces in the convergent portion by at least half of the maximum width in the last tenth of the distance moving from the maximum width limit to the second limit.

7. A mouthpiece as claimed in claim 1, in which the cross section of the middle portion is substantially diamond shaped.

8. A mouthpiece as claimed in claim 5, in which the outer periphery is substantially parallel with the width at the first and second limits and is substantially parallel with the second axis at the widest point.

9. A mouthpiece as claimed in claim 1, wherein the front portion includes an end wall which is set at an angle to the first axis such that a lower portion protrudes further along the first axis than an upper portion.

10. A mouthpiece as claimed in claim 1, wherein the rear portion includes an upper lip stop and a lower lip stop which are offset along the first axis such that the lower lip stop is set further away from the front portion than the upper lip stop.

11. A medical device comprising the mouthpiece claimed in claim 1.

12. An inhaler comprising the mouthpiece claimed in claim 1.
